(19) United States
(12) Patent Application Publication
(10) Pub. No.: US 2025/0087370 A1
(43) Pub. Date: Mar. 13, 2025

(54) COMPUTER-IMPLEMENTED METHOD AND DEVICE FOR GEOMETRICALLY DEFINING A COMPONENT ADAPTED TO AN ORGANISM UNIT

(71) Applicant: FRAUNHOFER-GESELLSCHAFT ZUR FÖRDERUNG DER ANGEWANDTEN FORSCHUNG E.V., München (DE)

(72) Inventor: Yannick LÖW, Hamburg (DE)

(21) Appl. No.: 18/580,277

(22) PCT Filed: Jul. 15, 2022
(86) PCT No.: PCT/DE2022/100507

§ 371 Date: Jan. 18, 2024

(30) Foreign Application Priority Data
Jul. 22, 2021 (DE) 10 2021 118 980.2

(51) Int. Cl. G16H 50/50; A61F 2/46; G16H 20/40; G16H 30/40

(52) U.S. Cl. CPC G16H 50/50; A61F 2/46; G16H 20/40; G16H 30/40; A61F 2002/4633

(57) ABSTRACT

The invention relates to a computer-implemented method (200) for geometrically defining a component (214) adapted to an organism unit, comprising the steps of: determining (202) at least one adaptation variable (222) by evaluating an image (210) of the organism unit, wherein the at least one adaptation variable (222) is based on a geometric characteristic of the organism unit, and defining (204) a component geometry (218) of the component (214) based on a component base geometry adapted with the at least one adaptation variable (222).

The invention relates to a computer-implemented method for geometrically defining a component adapted to an organism unit, a computer program product, a computer-readable data carrier and an apparatus for geometrically defining a component adapted to an organism unit.

Methods for geometrically defining a component are generally known. Components for organism units generally have a geometry that must be defined individually for each organism with which the component is to interact. For this purpose, the geometry of the organism unit, for example a hip joint or a finger bone, is usually reproduced. In addition, components for organism units are provided as standard sizes, for example shoes, glasses or prostheses, which generally do not meet all individual requirements.

Such components are currently essentially geometrically defined manually or are only individualized to a limited extent. A limited individualization of a component can be achieved, for example, by a Boolean subtraction of a 3-D scan from a predefined product. Components for healing or reducing cranial defects can be created using generative AI models, for example.

In addition, research is also looking into using AI or machine learning methods to define components as a generative model based directly on an image of the organism unit to be replaced. However, this approach is complex, as a large amount of training data is required. In addition, organism units and the components to be trained for them are such individual that the results of such a method generally do not provide satisfactory results and/or do not provide reproducible results. DE 10 2012 025 431 A1 discloses a method for detecting surface deformations of body surfaces in order to provide orthoses for body parts that are adapted to different load conditions.

It is a requirement for components adapted to organism units that they have an individualized design in order to enable increased accuracy of fit, increased comfort and/or improved force transmission. Furthermore, it is an aim to produce such components automatically and with high precision.

It is therefore an object of the invention to provide a computer-implemented method for geometrically defining a component adapted to an organism unit, a computer program product, a computer-readable data carrier and an apparatus for geometrically defining a component adapted to an organism unit, which reduce or eliminate one or more of the aforementioned disadvantages. In particular, it is an object of the invention to provide a solution which enables a component geometry to be defined in accordance with requirements with little effort.

This problem is solved with a computer-implemented method, a computer program product, a computer-readable data carrier and a device according to the features of the independent patent claims. Further advantageous embodiments of these aspects are given in the respective dependent patent claims. The features listed individually in the patent claims and the description can be combined with one another in any technologically meaningful way, with further embodiments of the invention being shown.

According to a first aspect, the problem is solved by a computer-implemented method for geometrically defining a component adapted to an organism unit, comprising the steps of: Determining at least one adaptation variable by evaluating an image of the organism unit, wherein the at least one adaptation variable is based on a geometric characteristic of the organism unit, and defining a component geometry of the component based on a component base geometry adapted with the at least one adaptation variable.

The component geometry is adapted to the organism unit. The component may, for example, be intended to replace or support the organism unit. It is particularly preferred that the component is a body replacement part, in particular an implant or a prosthesis. Furthermore, the body replacement part can be an orthosis or an exoskeleton.

In addition, the component may be a shoe, in particular with a medical purpose, or a pair of glasses, in particular electronic glasses. Furthermore, the component can generally be an organism-related, organism-connected, body-related and/or body-connected component which is adapted to the organism unit in operative connection with the component.

Among other things, the invention is based on the realization that a component geometry can be defined in an advantageous manner using a two-stage computer-implemented method. Furthermore, the invention is based on the realization that on the basis of a predefined basic component geometry, which is adapted to the organism unit with at least one adaptation variable, preferably two or more adaptation variables, a precise definition of the component geometry is possible with little effort. Furthermore, the component geometry defined in such a way is comprehensible for a user of the method, so that the disadvantage of geometries generated entirely on the basis of artificial intelligence is avoided or reduced.

The computer-implemented method makes it possible to automatically generate individualized component geometries from an image of an organism unit. This individualization has a positive effect on the functions of the component and leads in particular to increased accuracy of fit, increased comfort and/or improved power transmission.

It is preferred that the component is an implant and the component geometry is an implant geometry. An implant produced on the basis of such an implant geometry also leads to improved bone ingrowth and a longer product life. This in turn leads to the avoidance of cost-intensive and risky follow-up operations. This generally creates additional product added value. Furthermore, automation reduces costs and avoids human error, allowing for improved reproducibility of results.

The computer-implemented method also has the technical effect that the localization of the individual, characteristic points of the organism unit, namely by determining the at least one adaptation variable, and the design generation with explicit formulation of the solution space, namely by defining the component geometry, are largely separated. This means that specific requirements, norms and standards can be taken into account or adhered to.

Furthermore, the behavior of the definition of the component geometry is comprehensible and verifiable. This is in contrast to approaches that generate a component geometry directly on the basis of an image using AI. Furthermore, the component geometry can be further adapted or redesigned independently of the adaptation size.

In particular, the component is configured for an organism, for example the human body. The organism unit can be a bone or a joint, for example of a finger or a hip. In addition, the organism unit can be a head or a foot.

The image of the organism unit is a two-dimensional and/or three-dimensional image of the organism unit or a section of the organism unit. As will be explained in more detail below, the image of the organism unit can be a CT image, for example. The skilled person is also aware of further possibilities for creating an image of the organism unit.

The at least one adaptation variable is based on a geometric characteristic of the organism unit. In particular, the at least one adaptation variable represents a variable for adapting the basic component geometry, so that the component geometry of the component can be adapted to the organism unit. In particular, the adaptation variable describes the component geometry in combination with the basic component geometry. The geometric characteristic can be a macro- and/or micro-geometric characteristic. As will be explained in more detail below, the geometric characteristic can be, for example, a length, a cross-sectional geometry or a free-form surface definition. In principle, the adaptation variable is a variable that can be used to describe a geometric characteristic of the organism unit.

The component geometry is defined based on the basic component geometry adapted with the at least one adaptation variable. The basic component geometry can, for example, be a predefined basic component geometry for the organism unit. For example, the component base geometry can represent a finger joint geometry, a prosthesis geometry, an exoskeleton geometry or a shoe geometry.

In particular, the component base geometry is configured and provided in such a way that it can be adapted by means of the at least one adaptation variable. For example, the basic component geometry can be provided as a parameterizable initial model. The basic component geometry can also be understood as an initial geometry that can be individually adapted to the organism unit in question. Since, for example, a human finger joint is basically constructed in the same way, it can be described in principle with the basic component geometry, so that essentially only the individual formation of the human finger, which is at least partially replaced with the component, has to be taken into account.

A preferred embodiment of the computer-implemented method is characterized by the fact that the component base geometry is defined by at least one adaptation parameter, whereby the adaptation parameter for defining the component geometry is adapted by means of the adaptation variable. The adaptation parameter can, for example, relate to an extension from a distal end to a proximal end. This extension between the two ends is adapted during the definition of the component geometry by means of the adaptation parameter.

For example, the adaptation variable may relate to the extension mentioned above and the adaptation variable is based on the geometric characteristic that this extension is 25 mm. This makes it particularly easy to adapt the basic component geometry by means of the at least one adaptation size so that a suitable component geometry is defined.

A further preferred embodiment of the computer-implemented method is characterized by the fact that the at least one adaptation variable is based on a dimension and/or a position and/or location of the geometric characteristic. A dimension may be, for example, the extension between a distal end and a proximal end as mentioned above. A position of the geometric characteristic may be, for example, the position of a cross-section. The at least one adaptation variable can, for example, be determined by means of a localization algorithm. The at least one adaptation variable can also be based on two or more dimensions, for example two or three spatial directions. The position and location of the geometric characteristic can be determined relative to a reference point, for example.

In a further preferred embodiment of the computer-implemented method, it is provided that the step of defining the component geometry further comprises generating a directed graph based on the at least one adaptation variable. Further, the method may comprise the step of: Defining a component base geometry by generating a directed graph. For example, in the boundary representation scheme, the topology may be expressed in the form of an acyclic directed simple graph.

In particular, the directed graph comprises a set of nodes, also referred to as vertices, and a set of ordered pairs of nodes connected by edges. In particular, a three-dimensional geometry can be mapped with a directed graph, so that the component base geometry and/or the component geometry can be defined in an advantageous manner with the directed graph.

A further preferred embodiment of the computer-implemented method provides that the geometric characteristic of the organism unit is a one-, two- and/or multi-dimensional characteristic. A one-dimensional property can be, for example, an axis, a length, a dimension orthogonal to the axis and/or length or an attachment position of a muscle, a ligament or a tendon. Furthermore, the attachment position can also be described in two or more dimensions.

A two-dimensional property is, for example, a cross-sectional geometry. A multidimensional property can be, for example, the definition of a free-form surface. By describing the organism unit with one-, two- and/or multi-dimensional properties, a precise mapping can be made possible so that the component geometry can be precisely defined.

It is also preferred that the image is or comprises a surface model, preferably obtained using computed tomography. Furthermore, the image can be obtained using statistical shape models. Furthermore, the image can be obtained by a derivation from two-dimensional images, such as X-ray images.

A further preferred embodiment of the computer-implemented method comprises the step of: generating a digital component model based on the component geometry. The component model can, for example, serve as the basis for additive manufacturing of the component. It is preferred that the component model is a mesh-based representation, such as an STL model.

A further preferred embodiment of the computer-implemented method provides that the component geometry is further defined as a function of at least one functional requirement, a manufacturing requirement, a medical technology requirement and/or a certification requirement.

This design variant has the advantage that boundary conditions can be taken into account. Functional requirements can be, for example, the accommodation of further components, the accommodation of the actual component on or in an organism unit or the kinematics to other components or an organism unit. Manufacturing requirements can be, for example, minimum or maximum wall thicknesses. When producing the component by means of free-form surface production, in particular in a powder bed, the length and angle of overhangs can also be taken into account. Furthermore, minimum distances between two walls and powder removability must also be taken into account.

One medical technology requirement, for example, is the consideration of available tools, as drill sizes are standardized in medical technology, for example. Furthermore, distances from a sawing surface to a point worthy of medical preservation, such as a ligament attachment, can be taken into account.

A further preferred embodiment of the computer-implemented method comprises the step, in particular the iterative step: simulating at least one application situation of the component. It is also preferred that the component geometry is adapted based on the simulation results. This adaptation takes place in particular if such a need for adaptation has been detected by means of the simulation.

A simulated application situation can be, for example, a realistic load on the component applied in the organism, whereby, for example, strength is investigated. Such a simulation, also known as a strength simulation, enables the simulation of the load on the component in use, so that a statement on the fulfillment of requirements or an adaptation of the component geometry is advantageously possible on the basis of the simulation results obtained by such a simulation.

For example, this may involve reinforcing or slimming down individual sections of the component, in particular in accordance with the at least one adaptation variable. In addition, blood flow through the component can be simulated using a CFD simulation, for example.

A further preferred embodiment of the computer-implemented method is characterized in that the step of determining the adaptation variable is carried out by means of an algorithm which is based on training data comprising at least one learning image with at least one learning adaptation variable and determines correlations between at least the learning image and the learning adaptation variable.

It is particularly preferred that the algorithm determines correlations between the learning image, the learning adaptation variable and the image. In particular, the algorithm is a machine learning algorithm. The learning image is a predefined image, for example a CT image. The learning adaptation variable is an adaptation variable corresponding to the learning image. The learning image represents the input(s), the learning adaptation variable(s) the output(s) of the machine learning algorithm. The learning adaptation variables provided in the training data represent the correct function value to be learned for the respective learning image. After its initialization, the machine learning algorithm outputs the predicted learning adaptation variable and calculates the error for the correct learning adaptation variable. This error can be used to adapt the correlations of the learning algorithm. This procedure can be repeated iteratively until a sufficiently small error is obtained.

According to a further aspect, the object mentioned at the beginning is solved by a computer program product comprising instructions which, when the program is executed by a processor, cause the processor to perform the steps of the computer-implemented method according to one of the embodiments described above.

According to a further aspect, the problem mentioned at the beginning is solved by a computer-readable data carrier on which the computer program product according to the aspect mentioned in the preceding is stored.

According to a further aspect, the problem mentioned at the beginning is solved by an apparatus for geometrically defining a component, comprising a processor adapted to, upon execution of the computer program product according to the previous aspect by the processor, perform the steps of the computer-implemented method according to one of the embodiments described in the previous aspect.

It is further preferred that the device comprises a receiving unit. For example, the receiving unit may be adapted to receive the image of the organism unit. Furthermore, it is preferred that the device comprises a memory on which the image and/or data representing the component geometry can be stored.

Furthermore, the device may also comprise an output unit by means of which the component geometry can be output.

For further advantages, embodiment variants and embodiment details of the further aspects and their possible embodiments, reference is also made to the previous description of the corresponding features and embodiments of the computer-implemented method.

Preferred embodiments are explained by way of example with reference to the accompanying figures. The figures show

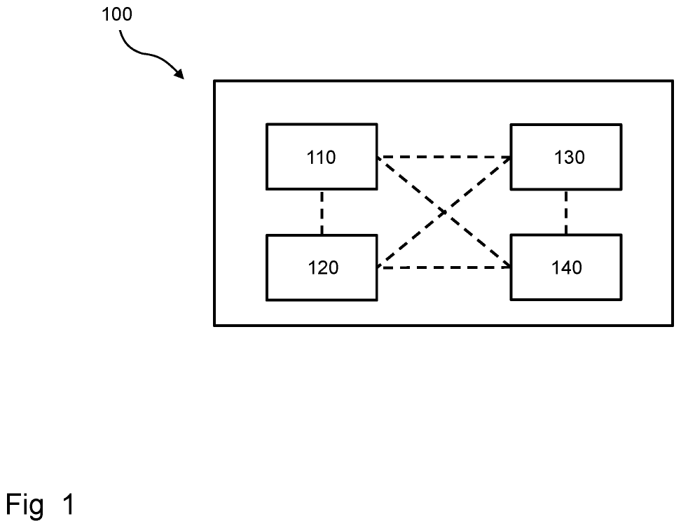

FIG. 1: a schematic view of an exemplary embodiment of an apparatus for geometrically defining a component;

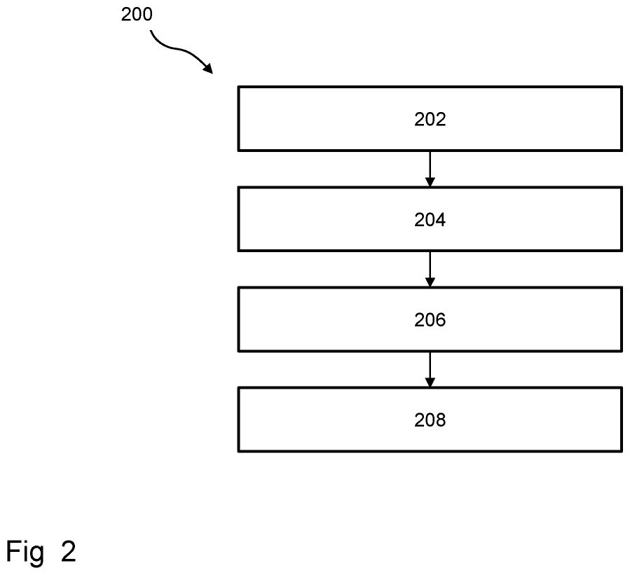

FIG. 2: a schematic view of an exemplary embodiment of a computer-implemented method for geometrically defining a component;

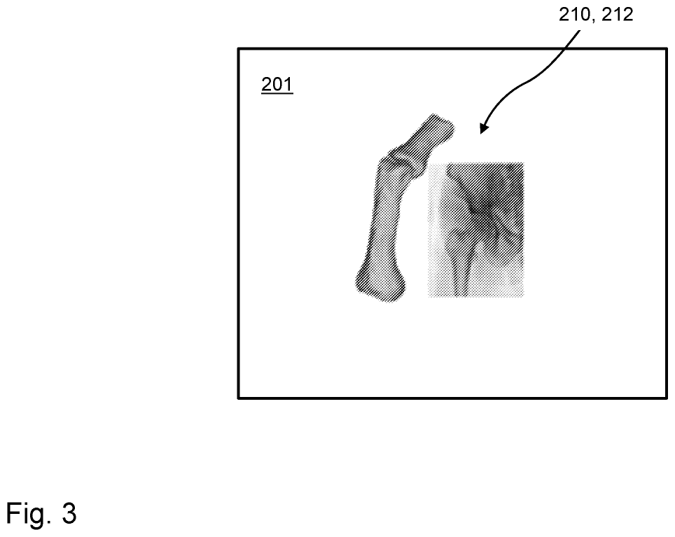

FIG. 3: a schematic view of an exemplary embodiment of a step for generating an image of the organism entity;

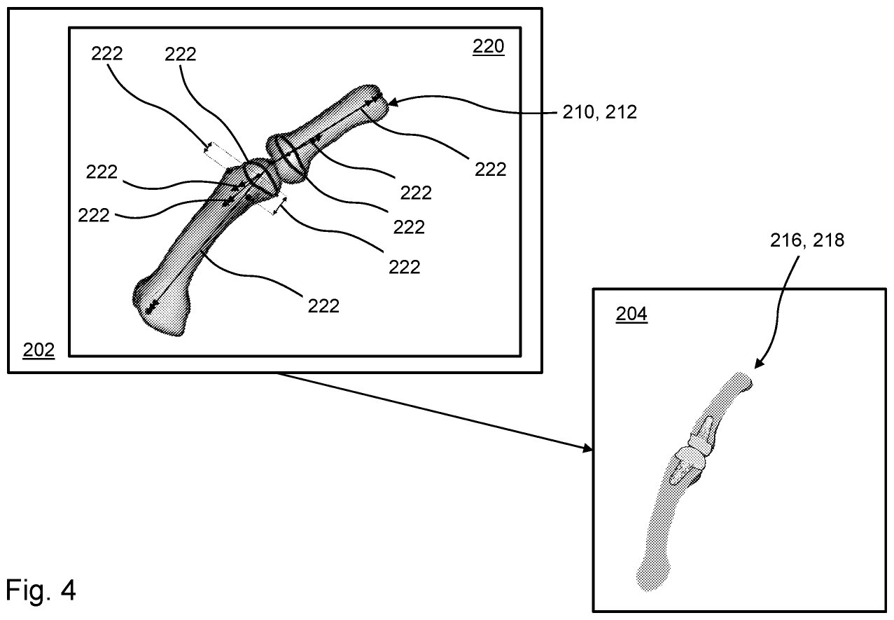

FIG. 4: a schematic view of an exemplary embodiment of two steps of the computer-implemented method; and

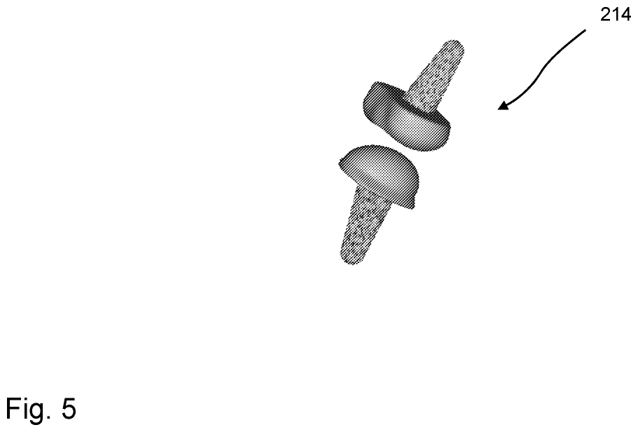

FIG. 5: a schematic view of an exemplary embodiment of a component.

In the figures, identical or essentially functionally identical or similar elements are designated with the same reference signs.

FIG. 1 shows the apparatus 100 for geometrically defining a component 214. The device 100 comprises a processor 120 adapted to perform the following steps of a computer-implemented method 200 when executing a correspondingly configured computer program product, namely determining 202 a plurality of adaptation variables 222 by evaluating an image 210 of an organism unit, wherein the plurality of adaptation variables 222 are based on geometric characteristics of the organism unit, and defining 204 a component geometry 218 of a component 214 based on a component base geometry adapted with the plurality of adaptation variables 222.

Further, the device 100 comprises a receiving unit 110, an output unit 130 and a memory 140. The receiving unit 110 may, for example, be configured to receive the image 210 of the organism unit. The image 210 may, for example, be obtained by means of a computer tomograph and provided to the receiving unit by suitable means. The image 210 can, for example, be stored at least temporarily in the memory 210. In addition, the defined component geometry 218 may also be stored in the memory 210. The output unit 130 preferably has access to the memory 210 and can provide the component geometry 218 or data characterizing the component geometry, for example to a manufacturing machine for additive manufacturing.

FIG. 2 shows a computer-implemented method 200 for geometrically defining a component 214 adapted to an organism unit 210. In step 202, a plurality of adaptation variables 222 are determined by evaluating an image of the organism unit 210. The adaptation variables 222 are based on characteristic geometric characteristics of the organism unit 210. The geometric characteristics include, for example, length, axes, cross-sectional geometries, and attachment points of tendons and muscles.

In step 204, a component geometry of the component 214 is defined based on a component base geometry adapted with the at least one adaptation variable 222. The component base geometry can be understood as a generalized, adaptable model of the component. Organism units of a healthy organism, for example the human body, essentially have a similar geometry. However, the specific geometric characteristics of the organism units vary from organism to organism. As a result, the basic component geometry must be adapted in order to achieve the best possible component geometry. This adaptation is performed by the determined adaptation variables 222.

In step 206, at least one application situation is simulated, wherein, for example, a strength simulation is performed. The simulation can be used to simulate different application situations, so that the component 214 produced on the basis of the defined component geometry is virtually tested before use. Furthermore, in step 206, the component geometry is adapted to the requirements based on the simulation results of the simulation, so that it can, for example, better absorb the loads that occur during use.

In step 208, a digital component model 216 is generated, wherein the digital component model 216 is based on or represents the component geometry. The digital component model 216 can, for example, be generated such that it represents the basis for producing the component 214 on which the component geometry 218 is based. For this purpose, the digital component model 216 can be an STL model, for example.

In FIG. 3, the generation of an image is shown as step 201. Here, an organism unit, in this case a finger joint and a hip joint, is captured using an imaging method. In particular, this is a three-dimensional representation as a result of an imaging method. The imaging method can be, for example, an X-ray method or a photograph, whereby the two-dimensional images obtained are preferably combined to form a three-dimensional image. Alternatively or additionally, the imaging method can be a computer tomography.

FIG. 4 shows two steps of the computer-implemented method. In step 202, a plurality of adaptation variables 222 are determined using an algorithm 220. The adaptation variables 222 are determined by evaluating an image 210 of the organism unit. The image 210 can be, for example, a surface model 212. The adaptation variables 222 are based on geometric characteristics of the organism unit, namely in particular lengths, axes and cross-sections. Subsequently, in step 204, the component geometry 218 is defined based on the adaptation variables 222 and then a component model 216 is created.

FIG. 5 shows a schematic view of an exemplary embodiment of a component 214 configured as an implant. The component geometry 218 of the component 214 has been defined using a computer-implemented method 200. This computer-implemented method comprises the steps of: Determining 202 a plurality of adaptation variables 222 by evaluating the image 210 of the organism unit, wherein the plurality of adaptation variables 222 are based on geometric characteristics of the organism unit, and defining 204 the component geometry 218 of the component 214 based on a component base geometry adapted with the plurality of adaptation variables 222.

The component base geometry may approximate the component geometry 218 of the component 214, wherein the component base geometry has been adapted to the specific formation of the organism unit using the plurality of adaptation variables 222.

The computer-implemented method described above enables the precise and application-oriented creation of component geometries 218 adapted to organism units, whereby the multi-stage structure of the method avoids the use of complex, error-prone and, in particular, non-reproducible or only partially comprehensible AI models.

The method is comparatively easy to implement on a computer and produces particularly advantageous results. Based on the generated component geometry 218, components can thus be produced that are more comfortable to wear, require fewer reoperations and are more economical.

### REFERENCE SIGNS

100 Device
110 Receiving unit
120 Processor
130 Output unit
140 Memory
200 Computer-implemented method
201 Generating an image
202 Determining at least one adaptation variable
204 Defining a component geometry
206 Simulating at least one application situation
208 Generating a digital component model
210 Image of an organism unit
212 Surface model
214 Component
216 Component model
218 Component geometry
220 Algorithm
222 Adaptation variables

1. Computer-implemented method for geometrically defining a component adapted to an organism unit, comprising the steps of:
Determining at least one adaptation variable by evaluating an image of the organism unit, wherein the at least one adaptation variable is based on a geometric characteristic of the organism unit, and
Defining a component geometry of the component based on a component base geometry adapted with the at least one adaptation variable.

2. Computer-implemented method according to claim 1, wherein the component is a body replacement part comprising an implant or a prosthesis.

3. Computer-implemented method according to claim 1, wherein the component base geometry is defined by at least one adaptation parameter, wherein the adaptation parameter is adapted to define the component geometry by means of the adaptation variable.

4. Computer-implemented method according to claim 1, wherein the at least one adaptation parameter is based on a dimension and/or a position of the geometric characteristic.

5. Computer-implemented method according to claim 1, wherein the step of defining the component geometry further comprises generating a directed graph based on the at least one adaptation variable.

6. Computer-implemented method according to claim 1, wherein the geometric characteristic of the organism entity is a one-, two- and/or multi-dimensional characteristic.

7. Computer-implemented method according to claim 1, wherein the image is or comprises a surface model.

8. Computer-implemented method according to claim 1, comprising the step of: generating a digital component model based on the component geometry.

9. Computer-implemented method according to claim 1, wherein the component geometry is further defined as a function of at least one manufacturing requirement, a medical technology requirement and/or a certification requirement.

10. Computer-implemented method according to claim 1, comprising the step of: simulating at least one application situation of the component and adapting the component geometry based on simulation results.

11. Computer-implemented method according to claim 1, wherein the step of determining the adaptation variable is performed by means of an algorithm which is based on training data comprising at least one learning image with at least one learning adaptation variable and determines correlations between at least the learning image and the learning adaptation variable.

12. Computer program product comprising instructions which, when the instructions are executed by a processor, cause the processor to perform the steps of the computer-implemented method according to claim 1.

13. Computer-readable data carrier on which the computer program product according to claim 12 is stored.

14. Apparatus for geometrically defining a component, comprising a processor adapted to, upon execution of the computer program product according to claim 12 by the processor, perform the steps of the computer-implemented method according to claim 1.
